(19) United States
(12) Patent Application Publication
(10) Pub. No.: US 2022/0398848 A1
(43) Pub. Date: Dec. 15, 2022

(54) Surveillance System and Method for Monitoring the Sterility of Objects in an Operation Room

(71) Applicant: Incremed AG, Zürich (CH)

(72) Inventors: Till Bay, Zürich (CH); Andreas Pedroni, Ennetbaden (CH); Mazda Farshad, Zumikon (CH)

(21) Appl. No.: 17/642,802

(22) PCT Filed: Sep. 14, 2020
(86) PCT No.: PCT/EP2020/075674

§ 371 Date: Mar. 14, 2022

(30) Foreign Application Priority Data
Sep. 13, 2019 (EP) 19197387.4

(51) Int. Cl. G06V 20/52; G16H 40/20; G06T 7/246; G06T 7/00; G06T 7/73; G06V 40/10; A61B 34/20

(52) U.S. Cl. CPC G06V 20/52; G16H 40/20; G06T 7/246; G06T 7/0012; G06T 7/73; G06V 40/10; A61B 34/20; G06T 2207/30196; G06T 2207/30232; G06T 2207/30204; G06V 2201/07; G06V 2201/03; A61B 2034/2057; A61B 2034/2065

(57) ABSTRACT

A method for monitoring and maintaining the sterility of objects in an operation room (1) is proposed, comprising: •registering target objects (3) and reference objects (4); •attributing to each target object (3) a set of reference objects (4); •attributing to each target object (3) a forbidden zone (90) and/or an allowed zone (91) based on a space occupied by the reference objects (4) attributed to this target object (3); tracking the target objects (3) and the reference objects (4); •determining, using data of the tracking, if a violation has occurred, the violation comprising that at least a part of a target object (3)—has entered the forbidden zone (90) attributed to that target object (3), and/or—has left the allowed zone (91) attributed to that target object (3). Furthermore, a surveillance system for performing this method is proposed.

### TECHNICAL FIELD

The present invention relates to a surveillance system and a method for monitoring and maintaining the sterility of objects, preferably of body parts, areas, and/or items, in an operation room.

### PRIOR ART

During an intervention, it is of critical importance that sterile persons, sterile fields, sterile instruments etc. remain sterile. Otherwise, there is an increased risk of an infection that could endanger the health of the patient.

In the operation room, there typically are two kinds of persons:
sterile persons that can get in contact with sterile fields and the sterile instruments; and
non-sterile persons that should not get in contact with a sterile person, fields, or instrument, for otherwise that person, field, or instrument is no longer considered sterile and needs to be exchanged or sterilized again.

Currently, it is common practice that the operation personnel, in particular the chief surgeon, monitors the sterility of the persons, areas, and items in the operation room. However, this method requires additional concentration by the personnel and is prone to mistakes.

US 2016/267327 A1 discloses a method for monitoring a patient within a medical monitoring area by means of a monitoring system with a depth camera device. The method generates a point cloud of the monitoring area with the monitoring system, analyzes the point cloud for detecting predefined objects, especially persons, then determined a location of at least one detected object in the monitoring area, and finally compares the determined location of the at least one detected object with at least one predefined value for the location of this detected object.

US 2012/075464 A1 discloses a further monitoring system including a camera adapted to capture images and output signals representative of the images. The camera may include one or more depth sensors that detect distances between the depth sensor and objects positioned within the field of view of the one or more cameras. A computer device processes the image signals and or depth signals from cameras and determines especially, whether an infection control protocol has been properly followed. The infection protocol can comprise identify objects in packages as being sterile. The system may further identify when sterile objects are removed from packaging and monitor what touches the sterile objects once they are removed from the packaging. On the other side, the system may also identify the location of a patient's dressing or open wound and monitor it such that only sterile objects approach this area. In both cases a local alert to warn the clinician of potential contamination may also be issued.

### SUMMARY OF THE INVENTION

In view of this state of the art, it is an object of the invention to provide systems and methods that can overcome disadvantages of the state of the art. In particular, systems and methods that allow for monitoring the sterility of objects in an operation room that preferably require less concentration from the personnel and/or allow for an improved monitoring are provided. These problems are solved by the surveillance system according to claim 1, and the method according to claim 10. Further embodiments are proposed in the dependent claims and the specification.

A surveillance system for monitoring—and preferably also for maintaining—the sterility of objects (e.g. body parts, areas, and/or items) in an operation room is proposed. The surveillance system comprises a tracking system that is designed for tracking at least one object in the operation room. The surveillance system is configured for registering objects. Such a registered object can e.g. be a body part (e.g. a body, an arm, and/or a head of a person), an area (e.g. a fixed sterile field), and/or an item (e.g. a surgical instrument), which preferably are (or at least can be) placed in the operation room. The registered objects form two sets of objects, a first set, whose elements shall be referred to as target objects, and a second set, whose elements shall be referred to as reference objects (each of these two sets comprising one or more objects). It is possible that an object is comprised in both sets, thus being a target object as well as a reference object. The surveillance system is further configured for attributing to each target object a subset of the set of reference objects (wherein the subset can be the full second set) and for attributing to each target object a forbidden zone and/or an allowed zone, wherein the forbidden zone resp. the allowed zone is based on the space occupied by the reference objects attributed to this target object. The tracking system is configured for tracking the target objects and the surveillance system is configured for determining (e.g. estimating)—using data provided by the tracking system—if a violation has occurred. The violation comprises, preferably is, that at least a part of a target object has entered the forbidden zone attributed to that target object, and/or has left the allowed zone attributed to that target object.

In some embodiments, the surveillance system comprises an output unit that is designed for outputting a signal, wherein the surveillance system is configured for outputting a signal via the output unit in case a violation has occurred. This can allow the personnel to take the necessary steps for maintaining and/or restoring the sterility of objects in the operation room.

The forbidden zone resp. allowed zone attributed to a target object typically is not fixed, but is adjusted based upon the space currently occupied by of the reference objects attributed to that target object. The movement of the reference objects are tracked by the tracking system, which allows for determining (e.g. estimating) the space currently occupied by the reference objects. In some cases, the position of a reference object is fixed (or is at least assumed to be fixed), for example, where the reference object is the area of a fixed operation table. Such fixed reference objects can e.g. be registered by inputting fixed coordinates or by an onetime measurement, and the tracking system tracks such fixed reference object e.g. in that the tracking system resp. the surveillance system knows the fixed position thereof and/or in that the tacking system measures data concerning a reference system (e.g. in cases where the tracking system is not fixed) for locating the fixed position within that reference system.

It can be the case that the forbidden zone attributed to a target object
either comprises the space occupied by the reference objects attributed to this target object (in an example, the forbidden zone attributed to a non-sterile person comprises the sterile operation table), or does not comprise any parts of the space occupied by the reference objects attributed to this target object (in an example, the forbidden zone attributed to a sterile surgeon does not comprise the sterile operation table).

It can be the case that the allowed zone attributed to a target object
either does not comprise any parts of the space occupied by the reference objects attributed to this target object (in an example, the allowed zone attributed to a non-sterile person does not comprise the operation table),
or comprises the space occupied by the reference objects attributed to this target object (in an example, the allowed zone attributed to a sterile surgeon comprises the operation table).

It can be the case that the forbidden zone resp. allowed zone of a target object is an environment of the reference objects attributed to this target object. An example of such an environment is a distance environment, which is determined by a fixed distance X from said reference objects in that everything that is at distance X or closer, e.g. in reality (3D) or in a projection onto a plane parallel to the floor (2D), to the reference objects is part of said X-environment. In a first example, an X-environment of a point according to reality is a ball of radius X whose center is this point; such environment shall be referred to as the ball-environment. In a second example, an X-environment of a point according to a vertical projection onto a plane parallel to the floor is a cylinder, whose basis is a disc parallel to a disc of radius X whose center is this point and that is parallel to the floor; such environment shall be referred to as the cylinder-environment.

It can be the case
that to some target objects a forbidden zone as well as an allowed zone is attributed;
that to some target objects only a forbidden zone, but no allowed zone is attributed; and/or
that to some target objects only an allowed zone, but no forbidden zone is attributed.

A forbidden zone can imply an allowed zone, namely the complementary of the forbidden zone. In the present text, many examples will address the forbidden zone and a violation occurring when the target object enters this forbidden zone. However, it is understood that—where applicable and with the necessary modifications—these examples could as well be described in terms of a complementary allowed zone and a violation occurring when target object leaves said allowed zone.

In an example, the reference objects attributed to a non-sterile body (the target object in this example) are all the sterile bodies in the operation room, and the forbidden zone attributed to this non-sterile body (target object) is defined as a 30-centimeter-environment around each of these sterile bodies (reference objects). The tracking system tracks the position of the sterile bodies (reference objects), and the surveillance system adjusts the forbidden zone according to the tracking of these sterile bodies (reference objects). The tracking system also tracks the position of said non-sterile body (target object) and if any part thereof has entered the forbidden zone, i.e. has moved within 30 centimeter of one of the sterile bodies (reference objects), a visual and/or acoustic signal is outputted to indicate the potential hygienic danger, so that the personnel can take the necessary measures.

It can be the case that the forbidden zone of a target object comprises a 25-centimeter-environment of the subset of reference objects attributed to it.

In some embodiments, the forbidden zone of a target object is comprised in a 45-centimeter-environment of the subset of reference objects attributed to it.

It can be the case that the forbidden zone of a target object
comprises a 25-centimeter-environment, preferably a 30-centimeter-environment, of the subset of reference objects attributed to it, and
is comprised in in a 45-centimeter-environment, preferably in a 40-centimeter-environment, of the subset of reference objects attributed to it.

A distance of ca. 25-45 centimeter, preferably of ca. 30-40 centimeter, can allow for avoiding an accidental transfer of microorganisms while still being practically manageable in an operation room.

It can be the case that an allowed zone for a target object is comprised in an environment of a reference object in form of an item. In other words, if the target object leaves said environment around this item (reference object), a violation occurs. For example, an allowed zone that is comprised in a 1-meter-environment of an operation table can be attributed to a surgeon, which allows for limiting the space that needs to be kept sterile.

In some embodiments, the surveillance system is configured for outputting different signals for indicating different kind of violations, e.g. depending on the degree of violation.

Preferably, the forbidden zone attributed to a target object is subdivided in sub-zones, and the outputted signal depends on which sub-zones the target object has entered.

In an example, the output unit is an acoustical output unit and the forbidden zone attributed to a target object is a 40-centimeter-environment of a reference object, the first subzone is a 30-to-40-centimeter-environment of that reference object and the second subzone is a 30-centimeter-environment of that reference object. If the target object is less than 40 centimeter, but not less than 30 centimeter from the reference object, a first signal is outputted at a first volume; and if the target object is less than 30 centimeter from the reference object, a second signal at a second volume that is higher than the first volume is outputted, thereby indicating the increased risk of contamination due to the increased proximity. Of course, the first and the second subzones of this example can be further subdivided, e.g. so that the output volume is a step-function depending on distance intervals.

In some embodiments, at least one parameter of the output signal, e.g. the volume of an acoustic output signal and/or the brightness of a visual output signal, is a function depending on a distance of a target object from a reference object attributed thereto. The function can e.g. be a step-function or a continuous function. As preferred, the function is a monotonous function, preferably a monotonous decreasing function. As preferred, the function is—at least in some environment of the distance zero—a strictly monotonously function, preferably a strictly monotonous decreasing function. In an example, the volume of an acoustic output signal and/or the brightness of a visual output signal is—at least in some environment of the distance zero—a strictly monotonously decreasing function of the distance; in other words: the closer the target object gets to the reference object, the louder resp. brighter the outputted signal is.

In some embodiments, the surveillance system outputs a signal in that it alters an existing signal. In an example, music is played in the operation room via an acoustic output unit, and in case a violation occurs, the surveillance system increases the volume and/or the speed of the music, and/or changes the pitch and/or the type of the music being played.

In some embodiments, the surveillance system comprises two or more output units, which preferably are located at two or more different positions within the operation room.

In some embodiments, the surveillance system is configured for outputting a signal that indicates the location in which a violation has occurred. According to a first example, the surveillance system is designed for outputting an acoustic signal that is modulated for appearing to originate—at least substantially—from the location in which the violation is occurring, e.g. by outputting a signal using a signal unit that is arranged at said location. According to a second example, the surveillance system is designed for outputting a visual signal that visually highlights—in reality and/or e.g. on a display—the location in which the violation is occurring.

In a further example, different signals are outputted depending on a group to which a violating target object belongs to, e.g. depending in whether the violation target object is a body part of a surgeon or a body part of a nurse. This can e.g. support the personnel in recognizing the cause of the violation faster.

In some embodiments, the surveillance unit is designed for outputting a parametric-modulated acoustic signal, i.e. a focused signal that can only be heard in a certain area.

The output unit preferably comprises an ultrasonic transducer. The area in which the acoustic signal is heard can be chose according to the tracked position of one or more objects, e.g. at the position of a target person that is causing a violation or at the position of a master user.

In some embodiments, the surveillance system is configured for recording data, preferably
data concerning
target objects,
reference objects,
attributed forbidden zones resp. allowed zones, and/or
data measured by the tracking system, preferably data concerning the tracking of objects (such as body parts, areas, and/or items).

The recorded data can be used to retrace the sterility status in the operation room later (e.g. in case an infection has occurred and/or in case of a liability investigation) and/or for training and analysis (e.g. for creating heat maps of objects of a certain sterility status, for optimizing the layout of operations rooms, and/or for training an artificial intelligence).

In some embodiments, the surveillance system comprises an object recognition unit that is designed for recognizing at least one object. The object recognition unit preferably comprises an artificial intelligence unit for recognizing the at least one object. Preferably, the surveillance unit comprises a data structure comprising data on the objects to be recognized. The surveillance system is preferably configured for automatically registering an object recognized by the object recognition unit.

In some embodiments, the tracking system is configured for tracking objects using data provided by the object recognition unit, e.g. by iteratively recognizing an object.

In some embodiments, the surveillance system, preferably an object recognition unit thereof, comprises a shape recognition unit that is designed for recognizing the shape of at least one item. Preferably, the surveillance system is configured for automatically registering an item recognized by the object recognition unit.

In some embodiments, the surveillance system comprises an object recognition unit in form of a person recognition unit that is designed for recognizing at least one person. The person recognition unit preferably comprises a face recognition unit that is designed for recognizing the face of at least one person. Alternatively or in addition, the person recognition unit can comprise a voice recognition unit that is designed for recognizing the voice of at least one person. Preferably, an artificial intelligence unit is comprised in the person recognition unit, the face recognition unit, and/or the voice recognition unit. A person recognition unit can be configured for recognizing a person using information retrieved from and/or linked to a marker attached to this person. Preferably, the surveillance system is configured for automatically registering a body part (in particular the full body) of a person recognized by the object recognition unit.

In some embodiments, the surveillance system is configured for automatically attributing an attribute to a recognized object. An attribute can be attributed in that a respective entry is made in a data structure. The information which attribute is to be attributed to an object can depend on entries in a data structure, and e.g. be linked to a class of an object (e.g. sterile object, surgeon, scalpel etc.) and/or to an individual object. An attribute of an object can e.g. be
that the object is a target object,
that the object is a reference object,
—for a target object—the subset of reference objects attributed thereto,
—for a target object—the forbidden zone attributed thereto,
—for a target object—the allowed zone attributed thereto,
that the object is member of a group,
a sterility status, and/or
—for a person (resp. full body)—that the person is a master user.

In an example, the surveillance system identifies a surgeon in the operation room using person recognition and based thereon automatically registers the surgeon and automatically attributes to it a forbidden zone, e.g. comprising all persons, areas, and/or items that are deemed non-sterile.

In some embodiments, the surveillance system comprises an object perimeter tracking unit that is designed for tracking, in particular at least estimating the position of, a perimeter of an object. The object perimeter tracking unit preferably comprises an artificial intelligence unit and/or uses an image-processing method, e.g. a foreground-background-subtraction process. A perimeter tracking unit can be beneficial for tracking (e.g. at least estimating) the space occupied by an object whose perimeter is not stable, such as a hose or a body.

In some embodiments, the surveillance system comprises an object perimeter tracking unit in form of a body perimeter tracking unit that is designed for tracking, in particular at least estimating the position of, a perimeter of a person's body. Recognizing a body perimeter can also allow for also recognizing and thereby tracking specific body parts other than the body itself, especially peripheral body parts such as for example the head and/or the hands/arms. A body perimeter tracking unit can therefore support determining the extent of a body part (in particular the body itself), and thus support determining
the space occupied by a body part (in particular the body itself) that is defined as a reference objects (which can allow for determining a forbidden zone resp. an allowed zone), and/or
if a body part (in particular the body itself) that is defined as a target object has entered the forbidden zone resp. has left the allowed zone attributed to that body part (which can allow for determining violations).

In some embodiments, the surveillance system comprises a skeleton tracking unit that is designed for tracking, in particular at least estimating the position of, the skeleton of a body. The skeleton tracking unit preferably comprises an artificial intelligence unit. Skeleton recognition can support determining the extent of a person's body. Recognizing a skeleton of a body can also allow for recognizing and thereby tracking specific body parts other that the skeleton resp. body itself, especially peripheral body parts such as for example the head and/or the hands/arms. A skeleton tracking unit can therefore support determining the extent of a body part (in particular the body itself), and thus support determining
the space occupied by a body part (in particular the body itself) that is defined as a reference objects (which can allow for determining a forbidden zone resp. an allowed zone), and/or
if a body part (in particular the body itself) that is defined as a target object has entered the forbidden zone resp. has left the allowed zone attributed to that body part (which can allow for determining violations).

In some embodiments, the surveillance system comprises an object recognition unit in form of a body part recognition unit that is designed for recognizing at least one body part.

The body part recognition unit preferably comprises a body perimeter tracking unit, a skeleton tracking unit, a shape recognition unit, and/or a marker.

It can be the case that the reference objects attributed to a target object comprises body parts, and that the forbidden zone resp. the allowed zone attributed to that target object is defined by the position of these body parts.

In an example, the target objects are the hands of a person and the reference objects attributed thereto comprise further body parts of that person, e.g. its waist-level, its armpits and/or its elbows. A violation occurs if at least one of the hand is positioned e.g.
below the level of a sterile field, especially of an operation table,
below his waist-level,
above his armpits, and/or
past his elbows.

A hand causing such violation can have an increased risk of becoming contaminated.

In another example, the head of person (e.g. a surgeon) is defined as a reference object and a violation occurs if the head is positioned over a sterile field (e.g. the operation table). Preventing positioning a head over a sterile field can decrease the risk of a contamination of the sterile field, e.g. by falling hair.

In some embodiments, the reference object is the front resp. the back of a person. The reference objects attributed to a front of a person (e.g. sterile person) can e.g. comprise the back of another person, but exclude the front of another person (e.g. another sterile person).

In some embodiments, the surveillance system, preferably the tracking system, comprises a facing direction recognition unit that is configured for recognizing the facing direction of a person, which can allow for determining in which direction the front resp. the back of a person is facing. The surveillance system is preferably configured for adjusting the forbidden zone and/or allowed zone of a target object depending on the facing direction of a person. It can e.g. be the case that the forbidden zone and/or allowed zone of a body part depends on the facing direction of the person to whom the body parts belongs.

In an example, the reference object attributed to a person's back is the operation table; and the forbidden zone attributed to this back is
a 30-centimeter-environment of the operation table, if the back is facing the operation table; and
empty (i.e. no forbidden zone is attributed to the back), if the back is not facing the operation table.

That means that in this case, that the monitoring of the back does not trigger a violation if the back is within the 30-centimeter-environment of the operation table while the person's front faces the operation table.

In some embodiments, the surveillance system comprises a data structure in which data about objects is stored. The data preferably comprises data that can support the recognition of an object (e.g. the voice of a person for identifying that particular person, or the shape of an item for identifying the type of that item). The surveillance system preferably is configured for automatically registering an object about which data is stored in the data structure and which is recognized by the surveillance system, e.g. by an object recognition unit thereof. The surveillance system preferably is configured for automatically attributing attributes to an object about which data is stored within the data structure and which is recognized by the surveillance system, wherein the attribution of the attribute is preferably based on data about this object (in particular about a class or a type of this object) stored in the data structure. The registration of an object and/or the attribution of an attribute to an object preferably comprises storing (in particular changing) respective data in the data structure. In an example, data on all registered objects are stored within the data structure. For each registered object it is stored if this object is considered a target object and/or a reference object. To each target object, the list of reference objects attributed thereto is stored, as well as the information if a forbidden zone or an allowed zone or both are attributed thereto and how the forbidden zone resp. allowed zone is determined based on the thereto attributed reference objects.

In some embodiments, the surveillance system comprises a processing unit. The processor is preferably configured for processing data and conduct calculations necessary for the functioning of the surveillance system, in particular for the functioning of an artificial intelligence unit. Preferably, the processing unit is connected to and/or comprises a data structure. The processing unit is preferably configured for processing data in connection with
registering objects,
attributing reference objects and the respective forbidden zone resp. allowed zone to a target object, and/or
determining if a violation has occurred.

In some embodiments, the surveillance system comprises an artificial intelligence unit that is configured for recognizing objects, persons, faces, facing directions, body parts, items, areas, shapes, skeletons, gestures, speech, and/or perimeters, preferably by using data measured by the tracking system and/or provided by a data structure. The artificial intelligence is preferably designed as a deep neural network. Preferably, the artificial intelligence unit is configured for using and/or interacting with image-processing methods, in particular image-processing algorithms.

The tracking system can comprise optical sensor means, i.e. sensor means using electromagnetic radiation in the visible-light spectrum. For example, the tracking system can comprise a wide-angle lens camera (whose angle of view is preferably 64 degrees or more), wherein the wide-angle lens camera is preferably a fisheye lens camera (whose angle of view is preferably 100 degrees or more). The camera is preferably positioned at a height of at least 2 meters from the floor, further preferred located at the ceiling, of the operation room. The surveillance system can be configured for processing, in particular for rectifying, the image taken by the wide-angle lens camera.

The tracking system can—in addition or alternatively—comprise infrared sensor means (i.e. sensor means using electromagnetic radiation in the infrared spectrum, in case of which the tracking system preferably also comprises an infrared emitter); radio-frequency sensor means (i.e. sensor means using electromagnetic radiation in the radio spectrum, for e.g. tracking RFID tags and/or a Bluetooth-Chips); and/or magnetic sensor means (i.e. using magnetic fields, e.g. sensor means for determining the position of an inertial measurement unit). The tracking system can comprise multiple sensors, the sensors being of the same type (e.g. all being optical sensors) or of different type (e.g. two optical sensors and one RFID sensor).

In some embodiments, the tracking system comprises a combination of a depth sensor that is designed for range imaging (e.g. using time-of-flight measurements, triangulation processes, and/or LIDAR measurements) and an artificial intelligence unit. An example of a device comprising such a combination is the AZURE KINECT DK system (by Microsoft). Such combination can be configured for acting as a skeleton tracking unit, an object perimeter tracking unit (in particular a body perimeter tracking unit, a shape recognition unit, and/or a body part recognition unit), facing direction recognition unit and/or a gesture command recognition unit. Preferably, this combination further comprises a RGB video camera, whose measured data can e.g. be used for recognizing markers. Optionally, the combination also comprises an inertial measurement unit (“IMU”) comprising an accelerometer and a gyroscope, which can allow for sensor orientation and spatial tracking. The proposed combination can be configured for acting as a face recognition unit and thereby acting as a person recognition unit. Alternatively or in addition, the person recognition unit can comprise a voice recognition unit that comprises a microphone and preferably an artificial intelligence unit. A microphone and an artificial intelligence unit can as well be comprised in a voice command recognition unit.

The surveillance system preferably comprises two or more different sensor technologies (e.g. optical sensors as well as radio-sensors) and/or use two or more different processing methods (e.g. a deep neural network process as well as a conventional foreground-background-subtraction process). This can allow for redundant tracking, which can allow for increasing the safety and/or the precision of the tracking and thus of the monitoring.

Different sensor technologies can also be used for measuring different types of objects, e.g. an optical sensor for shape recognition of an item and a radio-frequency sensor for tracking a certain body part to which a radio-chip-marker is attached.

In some embodiments, the surveillance system comprises at least one marker that is attached to an object and wherein the tracking system is designed for measuring data concerning a position of the at least one marker, and/or retrieving information from the marker. The measured data can be used for tracking the object to which the marker is attached. The retrieved information can be used for tracking, recognizing, and/or registering the object to which the marker is attached. Said information can e.g. comprise information
about the orientation of the marker and about its position and orientation relative to the position and orientation of the object to which it is attached (which can support tracking this object);
about the object to which it is attached (which can support tracking, recognizing, and/or registering this object).

The information can be directly stored within the marker and/or be stored in a data structure of the surveillance system, wherein the information is linked to the marker. Preferably, the surveillance system is configured to attribute at least one attribute to the object to which a marker is attached based on the information retrieved from that marker. Preferably, at least one marker is attached to each person that comprises at least one body part is to be registered by the surveillance system.

A marker can comprise a visual marker such as an image pattern, preferably an image pattern comprising a plurality of vertices and edges such as an ArUco-marker or a QR-marker. A visual marker attached to a person is preferably attached to a hat of that person, whereby its tracking by a visual tracking system arranged at a ceiling of the operation room can be supported. Different image patterns can allow for automatically identifying different groups of objects in the operation room.

A visual marker can comprise a colour marker, i.e. a marker that is not only in black and white. Using colours can support the recognition and the tracking of the marker, e.g. because the colour marker comprises a colour that is easy to track, e.g. because the chosen colour is atypical in an operation room. Preferably, the colour marker comprises red, orange, and/or violet. Different colour markers can be attached to different groups of objects (e.g. blue coloured markers to a group of sterile persons and red coloured markers to a group of non-sterile persons) which can support the surveillance system—and humans—in recognizing the category of an object (in particular of a person) easier.

In an example, a visual marker is attached to the head of a person. The tracking system is configured for tracking this marker and for retrieving information from this marker. The retrieved information is linked to information in a database to which the surveillance system has access, which allows the surveillance system to identify the person and that the hands of this person are to be registered, classified as a target object, and attributed with the attribute sterile. Based thereon, all non-sterile body parts are attributed to the hands as reference objects, and a 30-centimeter-environment of these reference objects is attributed to the hands as forbidden zone. The tracking system further comprises a body perimeter tracking unit that is designed for recognizing the perimeter of the person to which the marker is attached. The surveillance system estimates and in this sense tracks the position of the hands of that person based on the tracking of the perimeter of the body that is attached to the marker, preferably by using an artificial intelligence unit.

The marker can comprise a radio marker such as an RFID tag and/or a BLUETOOTH-Chip. A radio marker can e.g. be attached to a body part, thereby allowing for tracking this body part, e.g. a hand, a head, a waist, and/or an armpit.

In some embodiments, a class of master users that are authorized to give commands to the surveillance system is defined and registered with the surveillance system. A master user, e.g. a chief surgeon, can for example attribute attributes to objects registered with the surveillance system, e.g. if they are considered to be sterile or not. The categorization of being a target object and/or a reference object, as well as the attribution of reference objects and/or forbidden zones resp. allowed zones, can be based on such attributes. By attributing attributes to objects, the master user can manage the monitoring parameters during an intervention. Of course, a master user can as well be a target object and/or reference object, and the status of being a master user or not can be attributed as an attribute.

In some embodiments, the surveillance system comprises a command recognition unit for recognizing a command, e.g. a voice command recognition unit that is designed for recognizing a voice command and/or a gesture command recognition unit that is designed for recognizing a gesture command. Voice resp. gesture commands have the advantage that the command giver, e.g. a sterile person, can give the commands without touching anything, which otherwise could compromise his sterility. However, it is also possible that the command recognition unit comprises classical input devices such as a button, a mouse, and/or a keyboard, which e.g. are operated by a technician upon a verbal command by the chief surgeon. The surveillance system is preferably configured for registering master users (i.e. at least one master user), for recognizing a command given by a master user using the command recognition unit, and/or for executing the command that was given by the master user and was recognized by the command recognition unit.

A command preferably concerns attributing at least one attribute to an object. Possible commands given to the surveillance system by a master user can concern at least one of the following:
registering an object, in particular a body part (e.g. a full body), an area and/or an item,
defining an object as a target object and/or as a reference object,
attributing to a target object a subset of reference objects,
attributing to a target object a forbidden zone,
attributing to a target object an allowed zone,
attributing a target object to a group,
attributing to a target object a sterility status, and/or
attributing a person to the class of master users.

In an example, the chief surgeon is defined as the only master user and at least some registrations and/or attributions performed by the surveillance system require his consent. For instance, where a certain target object is to be attributed to a certain group (e.g. a group of sterile objects), the surveillance system sends a request concerning this attribution to the master user, e.g. by displaying a respective message on a display in the field of view of the master user. The master user then issues e.g. a gesture command, confirming that this target object shall indeed be attributed to this group. Upon recognizing the master user's command, the surveillance system attributes this target object to this group. Based on this membership, the surveillance system automatically attributes the subset of reference objects, and the forbidden zone resp. the allowed zone attributed to this group to this target object.

In some embodiments, the surveillance system is configured for recognizing at least some of the master users using person recognition, voice recognition, face recognition, and/or respective markers. Preferably, the surveillance system is configured for automatically registering a master user when it is recognized. In an example, the surveillance system knows, e.g. by using data provided by a data structure, that on this specific date an intervention is scheduled, that a certain person is scheduled to be the chief surgeon for this intervention is, and that the chief surgeon is defined to be a master user for this intervention. Upon the entering of this chief surgeon into the operation room, the surveillance system identifies this chief surgeon automatically (e.g. using face and/or voice recognition), and then automatically classifies the chief surgeon as a master user.

In some embodiments, the surveillance system is configured for requesting from a person that enters the monitoring range of the surveillance system to input data into the surveillance system, e.g. data concerning an attribute to be attributed to that person. The surveillance system preferably comprises a voice command recognition and/or a gesture command recognition unit for said data input. The data inputted by the person can be accepted by the system or subject to a confirmation request to a master user. In an example, the surveillance system recognizes that a person has entered its monitored range and requests the input of an ID-number and a sterility status of that person. Based on that input, the person is registered as a target object and/or a reference object (and attributed accordingly). After the person has left and re-entered the monitored range, the surveillance system automatically recognizes the person based on data collected during its first presence in the monitored range, and asks that person to confirm that its former sterility status is still applicable. Upon receiving the input that its hygienic status has changed, the surveillance system adjusts the reference objects/forbidden zone/allowed zone attributed to that person resp. to other objects according to that information. The surveillance system can be configured to treat all persons by default as non-sterile.

A method for monitoring—and preferably also for maintaining—the sterility of objects (e.g. body parts of persons, areas, and/or items) in an operation room is proposed. The method comprises:
registering a first set of objects, such objects being referred to as target objects, and a second set of objects, such objects being referred to as reference objects, wherein the objects preferably are body parts, areas, and/or items;
attributing to each target object a subset of the second set of reference objects;
attributing to each target object a forbidden zone and/or an allowed zone based on a space occupied by the subset of reference objects attributed to this target object;
tracking the target objects and the reference objects;
determining, using data of the tracking, if a violation has occurred, the violation comprising that at least a part of a target object
   has entered the forbidden zone attributed to that target object, and/or
   has left the allowed zone attributed to that target object.

The steps of the method can be taken in the order as stated above, or in any other technical reasonable order. In particular, at least some of these steps can be performed at least partly in parallel. Preferably, the method is performed using one of the surveillance systems described herein, wherein the step of tracking the target objects and the reference objects is preferably performed using the tracking system of this surveillance system,

In some variants, the method further comprises outputting a signal in case a violation has occurred. This can allow the personnel to take the necessary steps for maintaining and/or restoring the sterility of objects in the operation room. Preferably, the outputted signal indicates the location in which a violation has occurred, which ca allow the personnel to quickly identify the source of the violation.

In some embodiments of the surveillance system resp. of the method, for a group of two or more target objects
the subset of reference objects attributed to each target object of this group comprises, preferably consists of, a same subset of registered objects, and/or
the forbidden zone resp. the allowed zone attributed to each target object of this group comprises, preferably consists of, the same forbidden zone resp. the same allowed zone.

In an example, a group comprises only non-sterile target objects, e.g. wherein the group consists of all non-sterile body parts in the operation room, and the subset of reference objects attributed to each person in this group consist of
all body parts (if any) in the operation room that are considered sterile,
all areas (if any) in the operation room that are considered sterile, and
all items (if any) in the operation room that are considered sterile.

The forbidden zone attributed to each target object in this group is defined as an environment of this subset. This definition can allow monitoring that none of the non-sterile target objects comes close enough to the sterile body parts, sterile areas, and sterile items to possibly endanger their sterility.

In some embodiments of the surveillance system resp. of the method, a first group of target objects and a second group of target objects are defined, wherein
a common first subset of reference objects is comprised in each respective subset of reference objects attributed to a target object of the first group, and/or
a common second subset of reference objects is comprised in each respective subset of reference objects attributed to a target object of the second group.

Preferably,
the common first subset of reference objects comprises the second group of target objects, and/or
the common second subset of reference objects comprises the first group of target objects.

Preferably,
a common first forbidden zone resp. a common first allowed zone is comprised in each respective forbidden zone resp. allowed zone attributed a target object of the first group, and/or
a common second forbidden zone resp. a common second allowed zone is comprised in each respective forbidden zone resp. allowed zone attributed to a target object of the second group.

The membership to one of the two groups can e.g. be based on a two-valued attribute attributed to each target object, such as being considered sterile or not.

In an example, the target objects are divided into two groups, a first group consisting of all sterile body parts and a second group consisting of all non-sterile body parts, and
the subset of reference objects attributed to each non-sterile body part consists of all sterile body parts and all sterile fields in the operation room, and the forbidden zone attributed to each non-sterile body part is an environment (e.g. a 30-centimeter-environment) of this subset of reference objects; and
the subset of reference objects attributed to each sterile body part consists of all non-sterile body parts, and the forbidden zone attributed to each sterile body-part is an environment (e.g. a 30-centimeter-environment) of this subset of reference objects. The subset of reference objects attributed to each sterile body part can in addition comprise the operation table and to each sterile body part an allowed zone that comprises an environment (e.g. a 1-meter-environment) of the operation table can be attributed.

In some embodiments of the surveillance system resp. of the method, the forbidden zone and/or an allowed zone attributed to a target object depends on the facing direction of a person, wherein that person preferably
comprises at least one body part that is the target object, and/or
comprises at least one body part that is attributed to the target object as a reference object.

In some variants, the method comprises registering master users (i.e. at least one master user), recognizing commands given by a master user, and executing commands given by the master user.

In some variants, the method comprises automatically registering objects recognized by object recognition. For the recognition of persons, body parts and/or facing direction of persons, the object recognition preferably uses face recognition and/or voice recognition. The method preferably further comprises automatically attributing attributes to a recognized object.

Preferably, the methods described herein, or at least parts thereof, are realised as methods implemented in one of the surveillance systems described herein resp. as computer-implemented methods.

Furthermore, a computer program that comprises instructions to cause the execution of at least one of the herein described methods is proposed. In addition, a computer-readable medium having stored thereon at least one of said computer programs is proposed.

Furthermore, methods that are represented by the embodiments of the surveillance system disclosed herein and embodiments of the surveillance system designed for performing the methods disclosed herein are proposed.

A surveillance system for monitoring and maintaining the sterility of objects in an operation room comprises, as mentioned above, a tracking system designed for tracking a plurality of objects within the operation room. All these objects are registered in a database by the surveillance system. Such objects can be sterile objects; which can be in a package or separately. Such sterile objects can be instruments or part of the operation desk or operation surface. These objects can also be a person like a surgeon, and these objects can include the hands and the head of the surgeon be e.g. separately the hands or the head of the surgeon; and this at the same time. In other words, a surgeon can be registered as a (sterile) object, his back (not sterile), his head (sterile) and his hands (including the arm to the elbows sterile). The same registration applies beside movable objects as an instrument table, an instrument and e.g. the operation table. The registration is done in a database D comprising different tables, e.g. registering these objects as reference objects R and as target objects T. Each target object T is attributed a space value of a forbidden space F vis-à-vis each possible reference object R. It is also possible, but not mandatory to attribute a space value of an allowed space A vis-à-vis each possible reference object R. Preferably, if both values are stored the allowed space A and the forbidden space F complement one another for making up the space of the operation room.

Space value means a three-dimensional space within the surveillance perimeter in the operation room. The space value can span a distance cloud around an object as a specific minimum distance or be simply a parallelepiped (or usually rectangular cuboid) around the object in the room. The door or passage going outside of the operation room is considered forbidden space F for a sterile object/person and allowed space A for a non-sterile person/item. Anything or anyone entering the operation room via the door is considered initially non-sterile and attributed the corresponding space values vis-à-vis ail other registered items/persons as a target and as reference object (see below for a specific preferred handling of this case in an embodiment of the invention).

Each item, e.g. scalpel, catheter etc. is handled as a separate entry as target object T in the database D in view of each registered reference object R, but of course, the database entry of forbidden space F (or allowed space A if stored) a vis-á-vis the same reference objects R are identical for same type objects.

Now, over time t, the spatial relationship S between all target objects T and reference objects R is detected by the surveillance system.

The tracking system is configured for tracking the target objects and the reference objects on a 1:1 basis and the surveillance system is configured for determining if a violation has occurred. A violation is stated when at least a part of a target object T has entered the forbidden zone F attributed to that target object T in view of any reference object R in the operation room. In other words. The surveillance system checks the space values of all entries of target objects T in the room against any forbidden areas F of said target objects T vis-à-vis (i.e. relative to) of all reference objects R in the room based on the spatial relationship S. In other words, the forbidden areas F attributed to all reference objects R relative to a specific target object T sums up to an additive space value.

If allowed space A is used, the space value of the allowed zone A is the intersection of the space value of the sets of all reference objects R relative to this target object T. Then the surveillance system checks the space value of the target object T, if has left the allowed zone A attributed to that target object T vis-à-vis all reference objects R.

The advantage of the system is based on the use of an array of all n reference objects R₁ to Rₙ vis-à-vis all m target objects T₁ to Tₘ and the relative forbidden space value Fᵢⱼ for i=1 to n and j=1 to m, filling a database D(Rᵢ, Tⱼ, Fᵢⱼ) of persons/items in the room and their potential relationship.

The value for each combination of object at time t is there for e.g. 0=S(Rᵢ, Tⱼ, Fᵢⱼ, t), if the target object Tⱼ is not in the forbidden area Fᵢⱼ of reference object Rᵢ, and 1=S(Rᵢ, Tⱼ, Fᵢⱼ, t), if the target object Tⱼ is in the forbidden area Fᵢⱼ of reference object Rᵢ, Then, the technical result of the surveillance system is the sum of Σ_{i;j=1,1}^{n;m} S(Rᵢ, Fᵢⱼ, t) at a time t and any value greater or equal 1 is considered a violation, i.e. if at least one target object is in the forbidden area of at least one reference object.

In the case of the allowed zone embodiment, the value for each combination of object is there for e.g. 0=S(Rᵢ, Tⱼ, Aᵢⱼ, t), if the target object Tⱼ is in the allowed area Aᵢⱼ of reference object Rᵢ, and 1=S(Rᵢ, Tⱼ, Aᵢⱼ, t), if the target object Tⱼ is not in the allowed area Aᵢⱼ of reference object Rᵢ, Then, the technical result of the surveillance system is the sum of Σ_{i;j=1,1}^{n;m} S(Rᵢ, Tⱼ, Aᵢⱼ, t) and any value >=1 is considered a violation too, i.e. at least one target object has left all allowed areas of all reference objects at that time t.

Beside the easy handling of multiple objects, i.e. persons and items, on the same level, a further advantage of this approach is the possibility of checking more than the property of “pure” sterile character. A surgeon can be stored with his body parts, e.g. hands up to the arm pits and head and separately his back and front part. For a scalpel as target object can be taken in the hand of the surgeon as reference object, since both elements are sterile and there is no supplementary condition. And the head of the surgeon as reference object can be very close to the head of a different surgeon or nurse. But the head of the surgeon as target object would entire a forbidden area of the operation table as reference object, if he enters space above the operation table. The same can be considered true, if any hand/arm of a surgeon as target object leaves a specific space above the operation table as reference object. These violations can only be detected through spanning a database space of forbidden or allowed space values for each present object in a operation room vis-à-vis each other present object, i.e. a database of reference objects and target objects with corresponding forbidden spaces and attributed values.

It is possible to create different levels of alarm, based on the violation, e.g. an entry in the forbidden room of e.g. 10%, i.e. 3 cm when the cloud of forbidden space around an object is 30 cm, or 10 cm when the forbidden space from e.g. a table is 1 m.

One specific advantage of the method and system according to an embodiment of the invention can be realized when staff or items are entering the operation room, i.e. the database D can be modified on the fly. Beside the above mentioned position-based forbidden areas even in the case of sterile persons or items, every target and reference object is attributed the label sterile or non-sterile. An item or person can switch from sterile to non-sterile and a corresponding change of the forbidden area value happens, i.e. all non-positioned based forbidden spaces of sterile reference objects apply to this object as target object; or the other way round, the allowed space is reduced as reference object to the allowed space of non-sterile objects.

When a person, mostly a surgeon or nurse is entering the operation room, the person is considered to be non-sterile and is attributed the corresponding status and added to the database D as new target object T and as new reference object for all relevant existing target objects T. The surveillance system can at such entry sound an alarm which should trigger the stopping of the movement of said person who just entered for a predetermined time period. Usually the surgeon or nurse has already accomplished the necessary sterilization steps outside the room and wants to join the operation team. This stopping (which can be monitored by the surveillance system) and preferably a specific “entry” sound triggers the check by the already present hospital staff of the new person and after said specific time period, it is automatically acknowledged by the surveillance system that this new person is “sterile” in the sense that it has accomplished the necessary sterilization steps and this change will be registered in the database as a sterile surgeon.

The same procedure can apply when instruments on wheels or an instrument table is brought into the room. For the latter, the system can then identify each and every item on the table and register it as sterile. In case of non-identification of only one of the items, the surveillance system will notify the staff about the failure and the instrument should be leaving the room and re-prepared for a new entry.

### BRIEF DESCRIPTION OF THE DRAWINGS

Preferred embodiments of the invention are described in the following with reference to the drawings, which are solely for the purpose of illustrating the present preferred embodiments of the invention and not for the purpose of limiting the same. In the drawings,

FIGS. 1a, 1b show the current state of the art,

FIGS. 2a, 2b show a proposed surveillance system,

FIGS. 3a, 3b show an adjustment of a forbidden zone,

FIG. 4 shows an allowed zone and a forbidden zone,

FIGS. 5a, 5b show markers attached to a scrub head,

FIG. 5c shows a marker-less scrub head,

FIGS. 6a, 6b show a monitoring of body parts,

FIGS. 7a, 7b show an allowed zone for the hands of a sterile person,

FIG. 8 shows a forbidden zone for the head of a person,

FIG. 9 shows another monitoring of body parts,

FIG. 10a, 10b show a forbidden zone depending on facing direction,

FIG. 11a, 11b show an allowed resp. forbidden passing of two sterile bodies,

FIG. 12 shows giving a gesture command,

FIG. 13 shows object recognition,

FIG. 14 shows mutual attribution of two bodies,

FIG. 15 shows a processing unit,

FIG. 16a, 16b show indicating the location of a violation using signals,

FIG. 17 shows a flow diagram of a surveillance method for monitoring persons, and

FIG. 18 shows a flow diagram for commands given by a master user.

### DESCRIPTION OF PREFERRED EMBODIMENTS

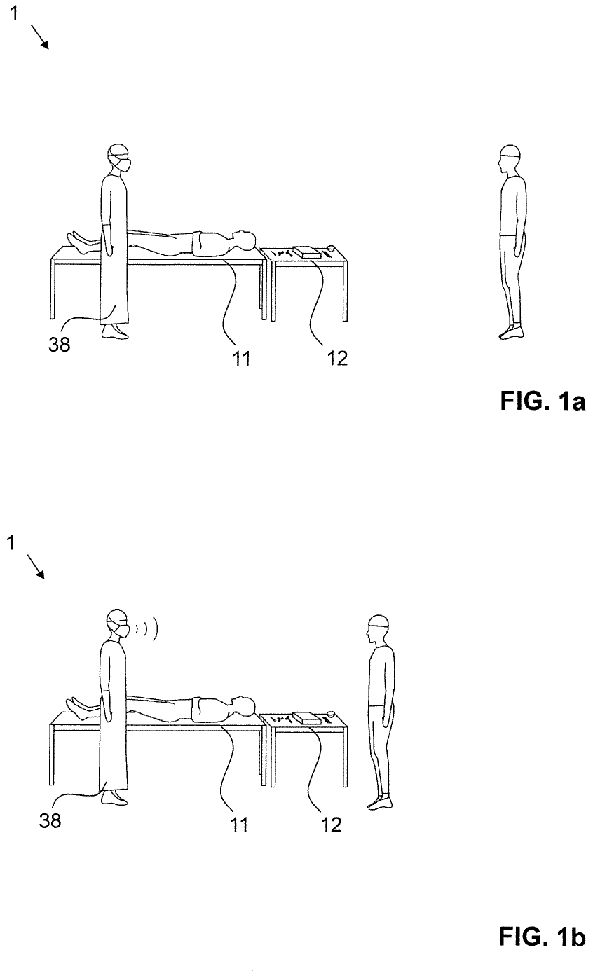

FIG. 1a shows an operation room 1 comprising sterile items such as an operation table 11 on which a patient is placed and an instrument table 12 on which instruments for use during an intervention are placed. In order for these items to remain sterile, none person considered non-sterile is allowed to come too close to these sterile items. Compliance with this rule is typically monitored by the chief surgeon 38 and/or the other personnel in the operation room 1. If, as shown in FIG. 1b, a non-sterile person comes too close to the sterile items, e.g. the chief surgeon 38 takes the necessary steps such as ordering the non-sterile person to distance itself from the sterile items or to replace resp. re-sterilize the now possibly contaminated items. However, this requires a lot of attention from personnel and this method is prone to failure.

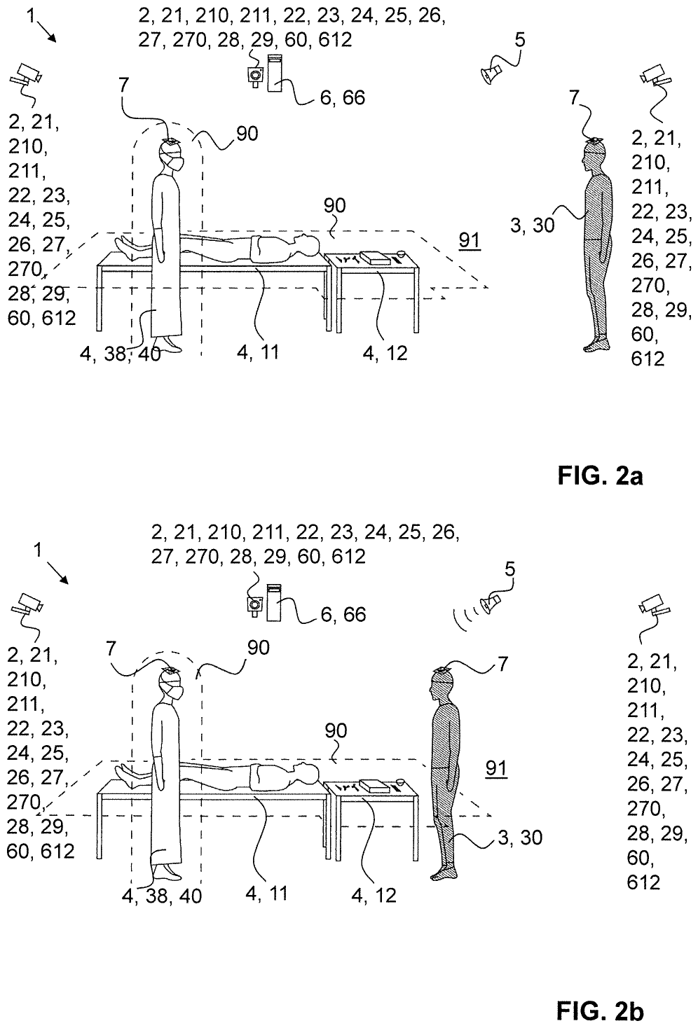

A surveillance system for automatically monitoring—and preferably also for maintaining—the sterility of objects, such as body parts, areas, and/or items, in an operation room 1, is proposed, an example of which is shown in FIG. 2a. The surveillance system is configured for attributing a forbidden zone 90 and/or an allowed zone 91 to a so-called target object 3, and is further configured for monitoring if the target object 3 stay out of its forbidden zone 90 resp. stays within its allowed zone 91. The forbidden zone 90 resp. allowed zone 91 is preferably defined using one or more so-called reference objects 4 that are attributed to the target object 3. Thereby it is possible to monitor the sterility of a sterile object by monitoring if it comes too close to a non-sterile object resp. if a non-sterile object comes too close to the sterile object.

The depicted surveillance system comprises a tracking system 2 that is designed for tracking objects in the operation room 1, preferably by at least quasi-continuously determining the position of these objects in the operation room 1. The tracking system 2 can for example comprise an AZURE KINECT DK system (by Microsoft), a DYNAMIC VISION SENSOR (by iniVation), and/or a SPECK sensor (by iniVation/aiCTX), each e.g. as available on Aug. 30, 2019. An AZURE KINECT DK system, which comprises a depths sensor 28, a camera 29, and an artificial intelligence unit 60, can e.g. be comprised in an object recognition unit 21, a body part person recognition unit 211, a shape recognition unit 22, a facing direction recognition unit 25, a skeleton tracking unit 26, an object perimeter tracking unit 27, a body perimeter tracking unit 270, and/or a gesture command recognition unit 612. A DYNAMIC VISION SENSOR can e.g. be comprised in an object recognition unit 21, a body part person recognition unit 211, a shape recognition unit 22, a facing direction recognition unit 25, a skeleton tracking unit 26, an object perimeter tracking unit 27, a body perimeter tracking unit 270, and/or a gesture command recognition unit 612. A SPECK sensor can e.g. be comprised in a face recognition unit 24, a person recognition unit 210, and/or a facing direction recognition unit 25.

The surveillance system is configured for registering objects 3, 4, preferably by using a processing unit 6 and/or a data structure 66. By registering an object 3, 4, that object 3, 4 becomes known to the surveillance system, so that the surveillance system can information-technological deal with this object 3, 4, e.g. store data concerning this object 3, 4 (e.g. data that allows recognition and/or tracking of this object 3, 4 and/or attributes attributed to this object 3,4). The objects 3, 4 preferably are body parts, areas, and/or items. Examples of a body part to be registered with the surveillance system is a person's full body, hand, arm, and/or head. Examples of an item to be registered with the surveillance system is an operation table 11 and/or an instrument table 12. An example of an area to be registered with the surveillance system is an area defined by an operation table 11 that is fixed (or at least assumed to be fixed), e.g. the area on and above the operation table. A registered object 3, 4 can be a target object 3, which is being monitored for violations, and at the same time be a reference object 4, which is used for defining the forbidden zone 90 and/or an allowed zone 91 for a target object 3.

In the following, multiple examples are described that concern target objects 3 in form of body parts, in particular full bodies; it is however understood that—where applicable and with the necessary modifications—these examples could as well be described for other target objects 3, such as items and/or areas.

The surveillance system is configured for attributing to each target object 3 a set of registered objects 4, and based thereon a forbidden zone 90 and/or an allowed zone 91. In the example of FIG. 2a, the target object 3, namely a full body of a first person 30, is considered non-sterile and the reference objects 4 attributed to it are two items, namely the operation table 11 and the instrument table 12, and one body part, namely a full body of a second person 40, e.g. the chief surgeon 38, each of which is considered sterile. In order to maintain the sterility of these sterile reference objects 4, a forbidden zone 90 that comprises a 30-centimeter-environment of the reference objects 4, i.e. the reference objects 4 themselves and everything that is within 30 centimeter or less thereof, is attributed to the target object 3. In the example of FIG. 2a, the depicted 30-centimeter-environment of the chief surgeon 38 is defined by the distance in all directions; and the depicted 30-centimeter-environment of the operation table 11 and the instrument table 12 is defined by a distance in a vertical projection onto a plane parallel to the floor (effectively defining a cylinder whose base is parallel to the polygon indicated by the broken line in the figure). Similarly, an allowed zone 91 for this target object 3 can be defined, e.g. as the complementary of the depicted forbidden zone 90. In this case, the allowed zone 91 for this target object 3 is everything but said 30-centimeter-environments of the attributed reference objects 4.

The tracking system 2 depicted in FIG. 2a is inter alia configured for recognizing objects by measuring data concerning the respective markers 7 attached to these objects. The markers 7 in this example are image patterns, which can be recognized using a camera 29 of the tracking system 2. The markers 7 allow the surveillance system to differentiate between the two depicted persons 3, 4, and to decide, e.g. using data stored in a data structure 66 of the surveillance system, that the one person is considered a target object 3 and the other person is attributed thereto as a reference object 4. In case two or more target objects 3 are to be monitored, the markers 7 can allow differentiating between these target objects 3, e.g. in case different reference objects 4, different forbidden zones 90, and/or different allowed zones 91 are attributed to different target objects 3. In addition or alternatively to markers 7, an object recognition unit 21, a person recognition unit 210, a body part person recognition unit 211, a shape recognition unit 22, a voice recognition unit 23, and/or a face recognition unit 24 can allow for recognizing the objects.

In addition, the tracking system 2 shown in FIG. 2a comprises an object perimeter tracking unit 27, which allows for tracking the perimeters of the respective objects 3, 4, which allows
determining the space currently occupied by the reference objects 4, which can lead to an adjustment of the attributed forbidden zones 90 resp. allowed zones 91; and/or
determining the space currently occupied by the target objects 3, which in turn allows for determining if a part of the target objects 3 has entered resp. has left a certain zone.

In case of body parts, in particular of full bodies, a skeleton tracking unit 26 can be used for estimating and this sense determining the space occupied by these body parts. In case of items, a shape recognition unit 22 can be used for determining the space occupied by these items.

The depicted surveillance system is further configured for determining (e.g. by estimating), by using data provided by the tracking system 2, if a violation has occurred, namely that a target object 3
has entered the forbidden zone 90 attributed to that target object 3, and/or
has left the allowed zone 91 attributed to that target object 3.

Such a violation is depicted in FIG. 2b, where the target object 3, i.e. at least a part of it, has entered its forbidden zone 90. In the depicted example, the surveillance system comprises an output unit 5 designed as a loudspeaker that outputs an acoustic signal indicating the sterility violation by the target object 3. In addition or alternatively, the output unit 5 can comprise a visual output unit such as
a display on which a warning message can be displayed (e.g. on a monitor and/or optical head mount display),
a warning light, and/or
a light beam emitter that allows for pointing at the location at which a violation has occurred.

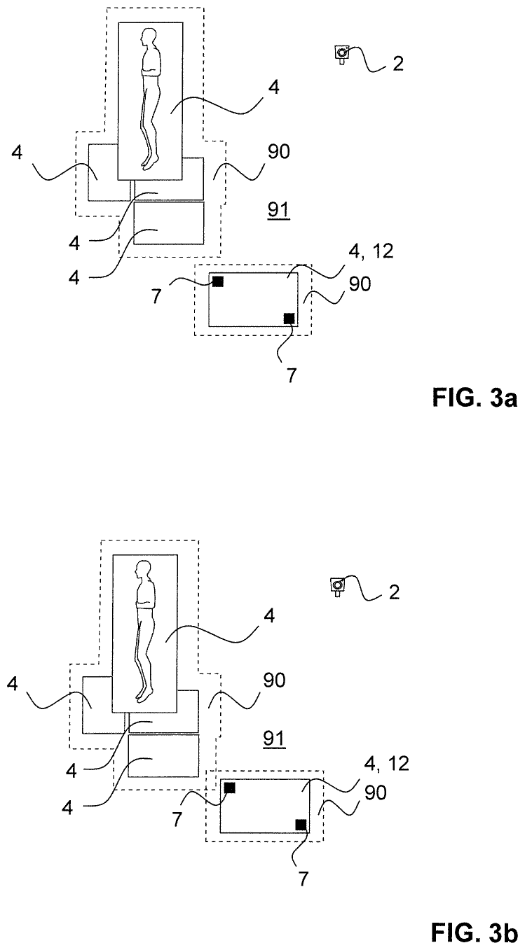

The forbidden zone 90 resp. allowed zone 91 attributed to a target object is preferably defined based on the space occupied by the reference objects 4 that are attributed to the target object, e.g. are an environment thereof. The tracking system 2 is preferably configured for tracking the reference objects 4, which allows for adjusting a forbidden zone 90 resp. an allowed zone 91 according to a displacement of the attributed reference objects 4. Such an example is shown in FIG. 3a and FIG. 3b, wherein the forbidden zone 90 is inter alia defined by a distance environment around the instrument table 12 and thus the forbidden zone 90 shown in FIG. 3a is adjusted according to the new position of the instrument table 12 in FIG. 3b. As described before, items, such as the instrument table 12, can be tracked using shape recognition. In the example of FIGS. 3a and 3b, markers 7 are attached to two diagonally opposed corners of the instrument table 12, which allows the tracking system 2 to track the position of the instrument table 12 by tracking the markers, e.g. in connection with data about the shape of the instrument table 12. As exemplified in FIG. 3a, the forbidden zone 90 can of course comprise multiple connection components.

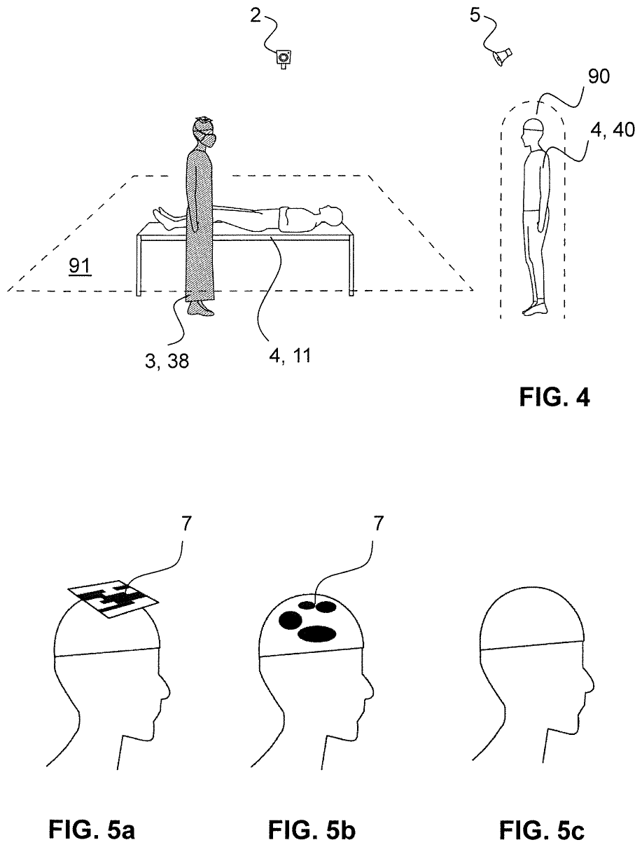

In the example shown in FIG. 4, the reference objects 4 attributed to a target object 3, in this case the full body of a chief surgeon 38 (who is considered sterile), are an operation table 11 (which is considered sterile) and a full body of a support person 40 (who is considered non-sterile). In the depicted example, a forbidden zone 90 attributed to the target object 3 is defined as a 30-centimeter-environment of the full body of the support person 40; and an allowed zone 91 attributed to the target object 3 is defined as an 1-meter-environment of the operation table 11. Of course, in this example the forbidden zone could instead as well be defined as to consist of the union of said 30-centimeter-environment of the non-sterile support person 40 and the complementary of said 1-meter-environment of the operation table 11.

The forbidden zone (resp. the allowed zone) attributed to each target object is preferably based on the space occupied by the reference objects attributed thereto in that either the space occupied by the reference objects is comprised in the forbidden zone (resp. allowed zone) or that no part of the space occupied by the reference objects is comprised in the forbidden zone (resp. allowed zone). However, the forbidden zone (resp. allowed zone) can e.g. also be based on the space occupied by the reference objects attributed to a target object in that it is the intersection and/or union of such zones and/or their complementary zones.

As shown in FIGS. 5a and 5b, markers 7 comprising an image pattern can be attached to the head of a person, which can be beneficial for its recognition using optical tracking systems that are installed at a height, such as a fisheye lens camera installed at a ceiling. As depicted in FIG. 5a, the marker 7 can comprise an image pattern printed on a dimensionally stable plate that is attached to a scrub. As shown in FIG. 5b, the image pattern 7 can also be printed on the scrub head. Two or more markers 7 can be different, which can allow for recognizing individual objects, classes of objects, attributes to be attributed to objects, and/or the position of the attached of marker. FIG. 5c shows an example where no such marker is used. In this case, the person can e.g. be recognized using a face recognition unit and/or a voice recognition unit, which possibly comprise an artificial intelligence unit.

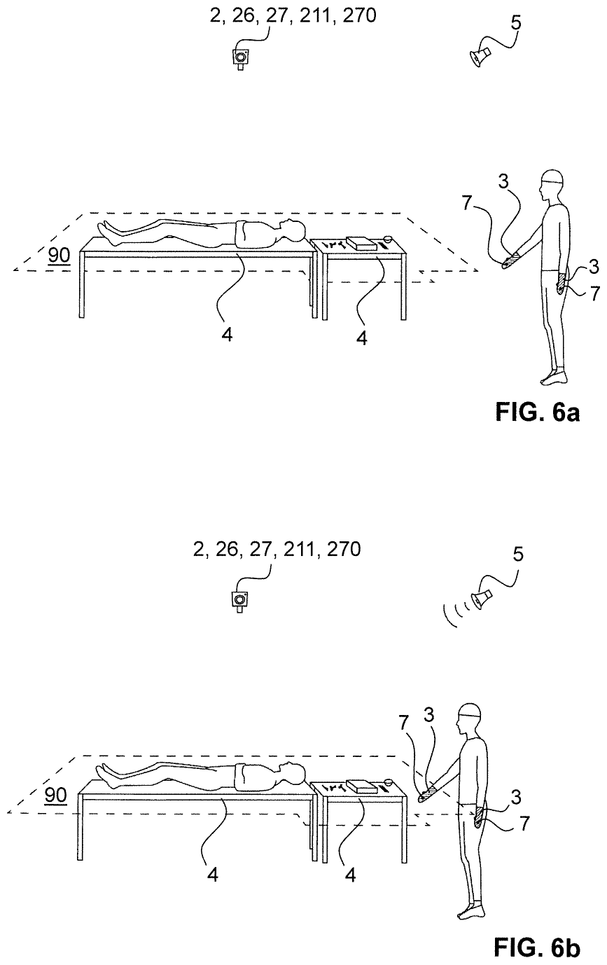

The surveillance system can be configured for monitoring target objects 3 in form of body parts as exemplified in FIG. 6a. In this example, radio markers 7, e.g. a RFID tag or a BLUETOOTH®-Chip, are attached to each of the body parts 3, in this case two hands, and the tracking system 2 is designed for tracking the radio markers 7, e.g. using a radio technology, and thereby the position of the body parts 3. In the depicted example, the same reference objects 4 and the same forbidden zone 90 are attributed to each hand 3, and if at least one of the hands 3 enters the forbidden zone 90, as depicted in the example of FIG. 6b, the output unit 5 indicates this violation.

As shown in FIGS. 6a, 6b, body parts, which can be target objects and/or reference objects, can be tracked by using tracking markers 7 attached thereto. Alternatively or in addition, the tracking system 2 can comprise an object perimeter tracking unit 27, in particular a body perimeter tracking unit 270, and/or a skeleton tracking unit 26, which can be used for tracking, e.g. at least estimating, the position of a body part, such as e.g. the arms/hands and/or the head of a person, possibly supported by a body part recognition unit 211.

In many cases, body parts other than the full body are monitored only for persons that are considered sterile, such as surgeons. Namely, while for non-sterile persons typically the whole body is considered non-sterile, for sterile persons it is efficient to only keep certain body parts, e.g. the hands and/or the front, sterilized; and thus is can be beneficial to monitor individual body parts of such a sterile person.

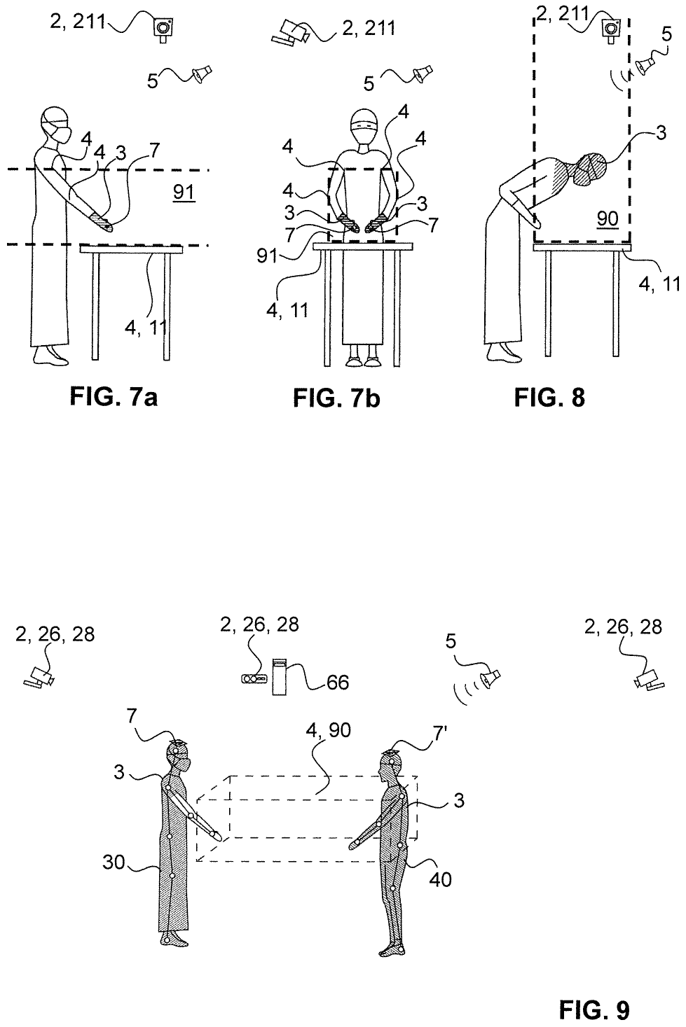

Body parts of a person can be reference objects 4 attributed to target objects 3 in form of other body parts of the same person. Such an example is shown in FIGS. 7a and 7b, wherein the allowed zone 91 attributed to a target object 3 in form of the hands of a person is a zone that is
bounded from below: by the level of a sterile field, such as that of an operation table 11,
bounded from above: by the level of the armpits of that person,
bounded from the sides: by the elbows of that person.

In particular, the allowed zone 91 attributed to the hands of the person in FIGS. 7a, 7b varies with the position of further body parts of that person, namely its armpits or elbows.

In the example shown in FIG. 8, the forbidden zone 90 for the head is the air domain above the operation table 11, while the same air domain is possibly comprised in an allowed zone of the hands of the person.

FIG. 9 shows an example where the reference object 4 is an area, which e.g. can be registered by inputting coordinates. The tracking system 2 tracks such a fixed reference object 4 e.g. in that the tracking system 2 resp. the surveillance system knows the fixed position thereof and/or in that the tacking system 2 measures data concerning a reference system for locating the fixed position of that area within that reference system, e.g. in cases where the tracking system 2 is not fixed and/or is restarted.

In the depicted example, two persons 30, 40 wear different markers 7, 7′ and from information retrieved from the markers 7, 7′, e.g. by consulting a data structure 66 in which information on the different markers 7, 7′ is stored, the surveillance system knows that of the first person 30 only the arms and hands are allowed to enter the area; and that the second person 40 is not allowed to enter the area at all. Therefore, the body of the first person 30 without the arms/hands and the full body of the second person 40 and are registered as target objects 3, and to both the area 4 is attributed as their respective forbidden zone 90.

The depicted tracking system 2 comprises a depth sensor 28 that supports the surveillance system in determining if something is positioned inside the area 4 or not; and a skeleton tracking unit 26 that supports the surveillance system in estimating the position of the respective skeleton of each of the persons 30, 40. Based on data about the respective skeleton, the surveillance system estimates the space occupied by the first person's 30 body minus its arms/hands and the space occupied by the second person's 40 full body, i.e. the space occupied by the respective target objects 3. Based on these estimates, the surveillance system estimates—and in this sense determines—if any of the target objects 3 have entered their respective forbidden zone 90, namely the area 4. If that is the case, e.g. because the second person 40 has entered the area 4 as shown in FIG. 9, the output unit 5 outputs a warning signal. This depicted scenario could e.g. be the case if the second person 40 is a non-sterile support person, the first person 30 is a sterile surgeon, and the area 4 is a sterile area, e.g. the area above the operation table (not shown in FIG. 9). The surveillance system can be configured for outputting different warning signals depending on which person has caused the violation.

In some cases, it may be beneficial to define that the set of reference objects, the forbidden zone, the allowed zone, and/or that the cases in which a violation occurs depend of further parameter, e.g. the facing direction of a person.

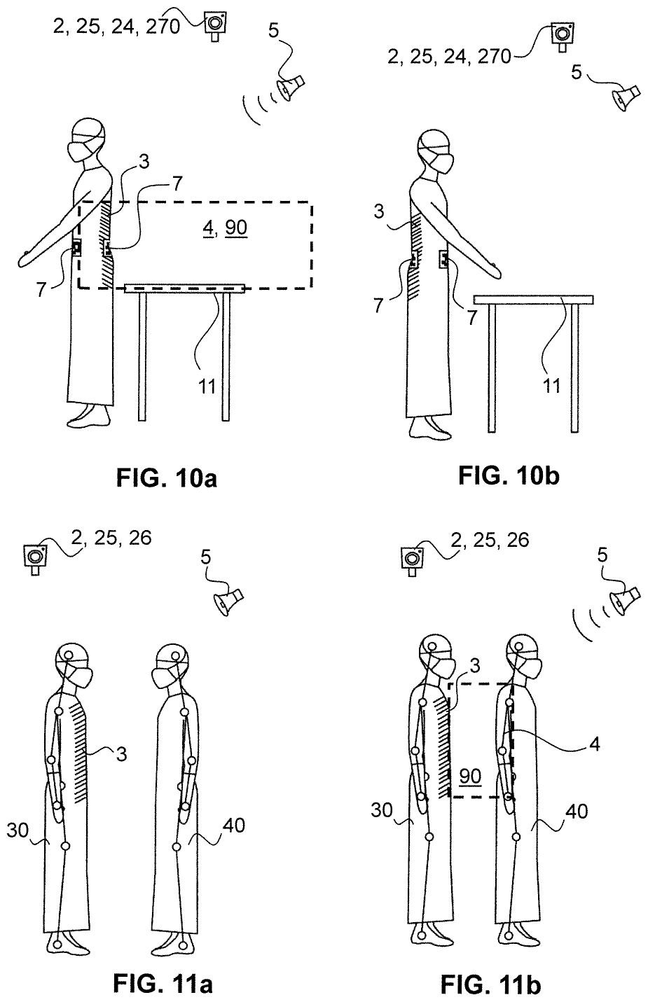

An example of such a case is depicted in FIGS. 10a and 10b, wherein the tracking system 2 comprises a facing direction recognition unit 25 that is designed for tracking the facing direction of a person, which can e.g. comprise a face recognition unit 24 and a body perimeter tracking unit 270. By recognizing the position of the face and the perimeter of the body, the surveillance unit can estimate, and in this sense determine, into which direction the person's front and back are facing. Alternatively or in addition, markers 7 can be attached to the front and/or the back of a person, whereby the facing direction recognition unit 25 can determine the direction to which the front resp. the back are facing. In the depicted case, the target object 3 is the back of the depicted person, to which—if, as shown in FIG. 10a, the back faces the operation table 11—the sterile area 4 is attributed as a forbidden zone 90; and to which—if, as shown in FIG. 10b, the back does not face the operation table 11—no forbidden zone is attributed.

FIGS. 11a and 11b exemplify a possibly monitoring for the passing of two sterile persons. For guaranteeing sterility, two sterile persons should preferably only pass front-to-front or back-to-back, but not face-to-back. In the depicted scenario, the target object 3 is the front of the first person 30 and if that front faces the front of the second person as shown in FIG. 11a, no reference object 4 resp. forbidden zone 90 is attributed to the front of the first person 30, which is why the two persons can pass each other that way without triggering a violation. However, if, as shown in FIG. 11b, the front of the first person faces the back of the second person, that second person's 40 back (as a reference object 4) and a forbidden zone 90 around that back is attributed to the front of the first person 30, which is why, if the two persons 30, 40 pass each other that way and in proximity to each other, a violation occurs. Of course, the front of the second person 40 can also be monitored as a target object in a similar manner.

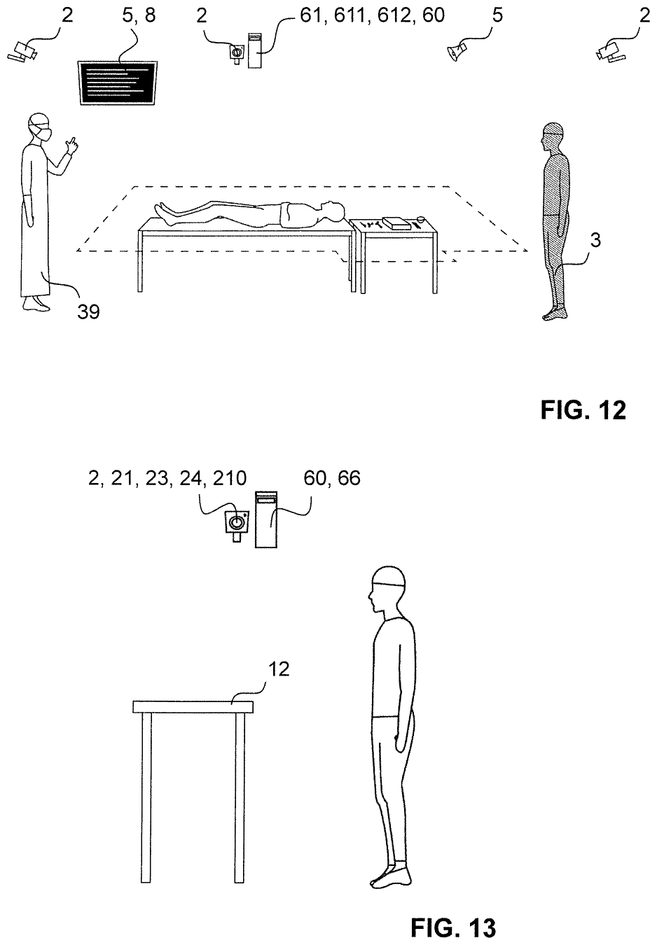

The surveillance system is preferably configured for registering one or more master users 39 and for executing processes based on commands given by these master users 39. As shown in FIG. 12, the surveillance system, e.g. the tracking system 2 thereof, can comprise a command recognition unit 61 designed to recognize a command of a master user 39. The command recognition unit 61 preferably comprises a voice command recognition unit 611, a gesture command recognition unit 612, and/or an artificial intelligence unit 60.

In the depicted example, a human target object 3 requests that an attribute, e.g. its sterility status, is changed, which possibly leads to the attribution of a new set of reference objects, a new forbidden zone, and/or a new allowed zone to at least one of its body parts. The surveillance system signals the request to the master user 39, e.g. via an acoustic output unit 5 or via an optical output unit 5 (e.g. a display 8). The master user 39 can react by giving a command, e.g. —as depicted here—a gesture command, thereby approving or rejecting the attribution of the new attribute to the target object 3. A display 8 for use with the surveillance system can be designed as a monitor (as shown in FIG. 12) or as a head-mounted display (not shown).

As shown in FIG. 13, the surveillance system, e.g. the tracking system 2 thereof, can comprise an object recognition unit 21 for recognizing objects, which can support the registration of recognized objects. The object recognition unit 21 can for example comprise an artificial intelligence unit 60 that is designed for identifying objects based on an analysis of an image of the objects. In the depicted example, the artificial intelligence unit is trained to recognize the instrument table 12 and is thereby able to recognize and register the instrument table 12 automatically. Preferably, the surveillance system is configured for automatically attribute attributes to recognized objects, e.g. based on entries in a data structure 66.

In addition or alternatively, the surveillance system can use markers (not shown in FIG. 13) for recognizing and registering an object, possibly by additionally using information about the markers, the objects, and/or their relative placement.

In addition or alternatively, the surveillance system can be designed for registering objects based on user input, e.g. inputted via a mouse, a keyboard, a touchpad, a touchscreen, and/or gesture commands. Similarly, attributes can be attributed via user input.

The object recognition unit 21 can in particular be designed as a person recognition unit 210, which can allow for recognizing individual persons. The person recognition unit 210 can e.g. use biometrical data of a person, a password, and/or a marker for recognizing a person. Preferably, the surveillance system is configured for automatically registering the recognized person resp. a body part thereof e.g. as a target object, as a reference object and/or as a master user. A person recognition unit 210 can for example comprise a voice recognition unit 23 and/or a face recognition unit 24.

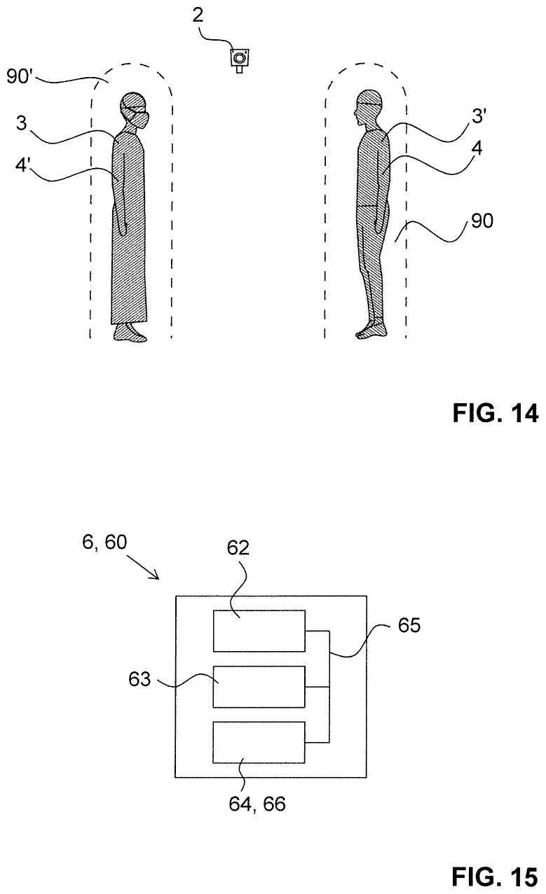

In the example of FIG. 14, a first human body 3 and a second human body 3′ are registered as target objects. To first body 3 the second body 3′ is attributed as a reference object 4, and based thereon an environment of the second body 3′ is attributed to the first body 3 as its forbidden zone 90. Furthermore, to the second body 3′ the first body 3 is attributed as a reference object 4′, and based thereon an environment of the first body 3 is attributed to the second body 3′ as its forbidden zone 90′. This can e.g. be the case where the first person 3 is considered sterile and the second person 3′ is considered non-sterile, so that the two should not get close to each other.

FIG. 15 shows a processing unit 6 as it can be used in the surveillance system for calculations, in particular in an artificial intelligence unit 60 of the surveillance system. The displayed processing unit 6 comprises a processor (CPU) 62 and a volatile (e.g. RAM) memory 63 and/or a non-volatile (e.g. a hard disk) memory 64, wherein the processor 62 communicates with the memory modules 63, 64 using one or more data buses 65. Preferably, the non-volatile memory 64 comprises a data structure 66 and/or the processing unit is connected to a data structure.

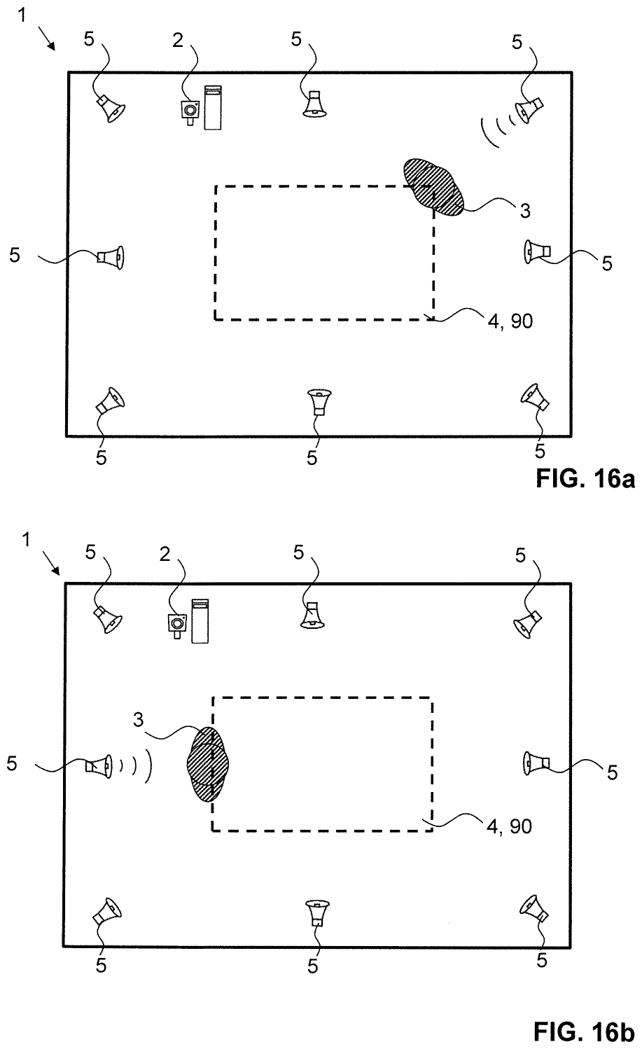

In some embodiments, the surveillance system is configured for outputting a signal that indicates the location in which a violation occurs. Such an example is shown in FIGS. 16a and 16b, wherein the depicted surveillance system comprises multiple loudspeakers 5, each being arranged at a different location of the operation room 1. In case a violation occurs, the acoustical signal is outputted by the loudspeaker(s) closest to the location of the violation, thereby allowing the personnel to identify the cause of the violation quickly.

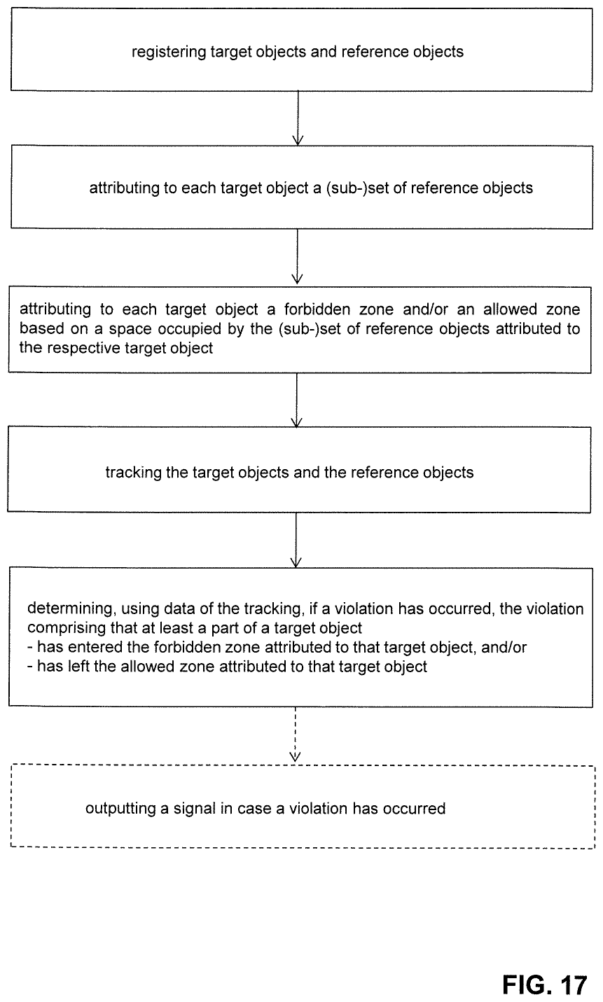

FIG. 17 shows a flow diagram of a proposed method for monitoring—and preferably also for maintaining—the sterility of objects in an operation room, comprising the steps of:
registering target objects and reference objects;
attributing to each target object a (sub-)set of reference objects;
attributing to each target object a forbidden zone and/or an allowed zone based on a space occupied by the (sub-)set of reference objects attributed to the respective target object;
tracking the target objects and the reference objects;
determining, using data of the tracking, if a violation has occurred, the violation comprising that at least a part of a target object
has entered the forbidden zone attributed to that target object, and/or
has left the allowed zone attributed to that target object.

The method optionally comprises outputting a signal in case a violation has occurred.

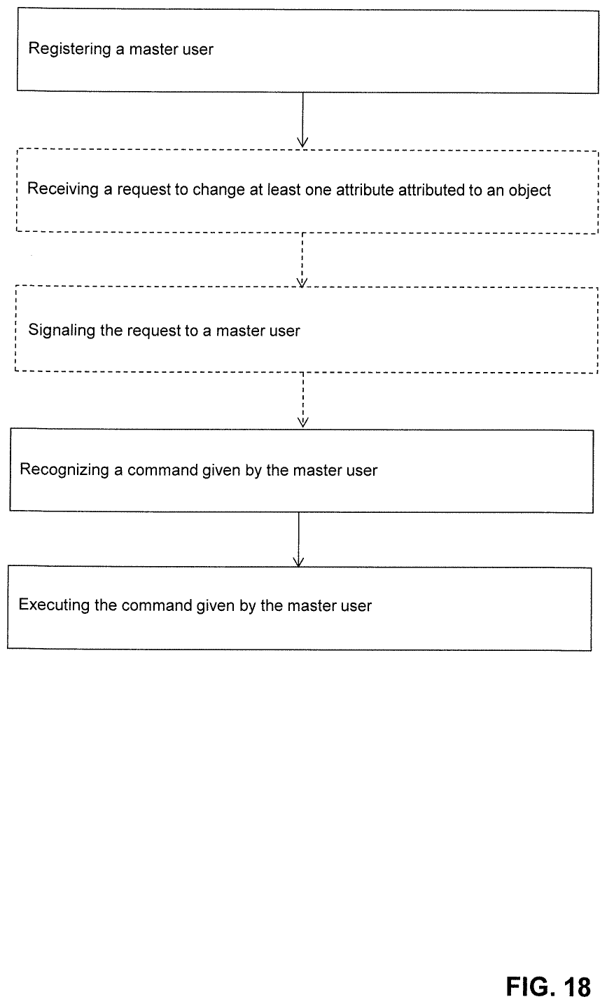

FIG. 18 shows a flow diagram for a method comprising the steps of:
registering a master user,
recognizing a command given by the master user, and
executing the command given by the master user.

As shown in FIG. 18, the method can optionally comprise receiving a request to change at least one attribute attributed to an object, and signalling the request to a master user.

Of course, the order of executing the steps of the proposed methods can vary in any technically useful manner, including parallel execution.

LIST OF REFERENCE SIGNS

| | |
|---|---|
| 1 | operation room |
| 11 | operation table |
| 12 | instrument table |
| 2 | tracking system |
| 21 | object recognition unit |
| 210 | person recognition unit |
| 211 | body part recognition unit |
| 22 | shape recognition unit |
| 23 | voice recognition unit |
| 24 | face recognition unit |
| 25 | facing direction recognition |
| | unit |
| 26 | skeleton tracking unit |
| 27 | object perimeter tracking unit |
| 270 | body perimeter tracking unit |
| 28 | depth sensor |
| 29 | camera |
| 3 | target object |
| 30 | person |
| 38 | surgeon |
| 39 | master user |
| 4 | reference object |
| 40 | person |
| 5 | output unit |
| 6 | processing unit |
| 60 | artificial intelligence unit |
| 61 | command recognition unit |
| 611 | voice command recognition |
| | unit |
| 612 | gesture command recognition |
| | unit |
| 62 | CPU |
| 63 | volatile memory |
| 64 | non-volatile memory |
| 65 | bus |
| 66 | data structure |
| 7 | marker |
| 8 | display |
| 90 | forbidden zone |
| 91 | allowed zone |

1.-15. (canceled)

16. Surveillance system for monitoring and maintaining the sterility of objects in an operation room (1),
comprising a tracking system (2) designed for tracking at least one object within the operation room (1);
wherein the surveillance system is configured for:
registering a first set of objects, these objects being referred to as target objects (3), and a second set of objects, these objects being referred to as reference objects (4), wherein the objects (3,4) preferably are body parts, areas, and/or items,
attributing to each target object (3) a subset of the second set of reference objects (4), and
attributing to each target object (3) a forbidden zone (90) and/or an allowed zone (91) based on a space occupied by the subset of reference objects (4) attributed to this target object (3);
wherein the tracking system (2) is configured for tracking the target objects (3) and the reference objects (4); and
wherein the surveillance system is configured for
determining, using data provided by the tracking system (2), if a violation has occurred, the violation comprising that at least a part of a target object (3):
has entered the forbidden zone (90) attributed to that target object (3), and/or
has left the allowed zone (91) attributed to that target object (3).

17. The surveillance system of claim 16, wherein the surveillance system further comprises an output unit (5) and wherein the surveillance system is configured for outputting a signal via the output unit (5) in case a violation has occurred.

18. The surveillance system of claim 17, wherein the surveillance system is configured for outputting a signal that indicates the location in which a violation has occurred.

19. The surveillance system of claim 16, wherein the surveillance system comprises at least one marker (7) that is attached to an object (3,4) and wherein the tracking system (2) is designed for measuring data concerning a position of the at least one marker (7), and/or for retrieving data from the marker (7).

20. The surveillance system of claim 16, wherein the surveillance system comprises an object recognition unit (21) designed for recognizing at least one object (3,4), wherein the at least one object (3,4) preferably is a person, a body part, an area, and/or an item.

21. The surveillance system of claim 20, wherein the surveillance system comprises a data structure (66) in which data about objects (3,4) is stored, and wherein the surveillance system is configured for automatically
registering an object (3,4) about which data is stored within the data structure (66) and which is recognized by the object recognition unit (21), and/or
attributing attributes to an object (3,4) about which data is stored within the data structure (66) and which recognized by the object recognition unit (21), wherein the attribution of the attribute is based on data about this object (3,4) stored in the data structure (66).

22. The surveillance system of claim 16, wherein the surveillance system comprises a facing direction recognition unit (25) that is configured for recognizing the facing direction of a person (30,40), and wherein the forbidden zone (90) and/or an allowed zone (91) attributed to a target object (3) depends on the facing direction of the person (30,40), wherein the person (30,40) preferably
comprises at least one body part that is the target object (3), and/or
comprises at least one body part that is attributed to the target object (3) as a reference object (4).

23. The surveillance system of claim 16, comprising a command recognition unit (61) designed for recognizing commands, wherein the surveillance system is configured for
registering master users (39),
recognizing commands given by a master user (39) using the command recognition unit (61), and
executing the commands given by the master user (39).

24. The surveillance system of claim 16, comprising a processing unit (6), wherein the processing unit (6) preferably is configured for processing data in connection with registering objects (3,4),
attributing reference objects (4) and the respective forbidden zone (90) resp. allowed zone (91) to a target object (3), and/or
determining if a violation has occurred.

25. Method for monitoring and maintaining the sterility of objects in an operation room (1), using the surveillance system of claim 16, the method comprising:
registering a first set of objects, such objects being referred to as target objects (3), and a second set of objects, such objects being referred to as reference objects (4), wherein the objects (3,4) preferably are body parts, areas, and/or items;
attributing to each target object (3) a subset of the second set of reference objects (4);
attributing to each target object (3) a forbidden zone (90) and/or an allowed zone (91) based on a space occupied by the subset of reference objects (4) attributed to this target object (3);
tracking, using the tracking system (2) of the surveillance system, the target objects (3) and the reference objects (4);
determining, using data of the tracking, if a violation has occurred, the violation comprising that at least a part of a target object (3)
has entered the forbidden zone (90) attributed to that target object (3), and/or
has left the allowed zone (91) attributed to that target object (3).

26. The method of claim 25, comprising outputting a signal in case a violation has occurred,
preferably wherein outputted signal indicates the location in which a violation has occurred.

27. The method of claim 25, wherein the forbidden zone (90) resp. the allowed zone (91) attributed to a target object (3) satisfies exactly one of the following properties:
it comprises the space occupied by the subset of reference objects (4) attributed to this target object (3),
it does not comprise any part of the space occupied by the subset of reference objects (4) attributed to this target object (3).

28. The method of claim 25, wherein a first group of target objects (3) and a second group of target objects (3) are defined, wherein
a common first subset of reference objects (4) is comprised in each respective subset of reference objects (4) attributed to a target object (3) of the first group, and/or
a common second subset of reference objects (4) is comprised in each respective subset of reference objects (4) attributed to a target object (3) of the second group.

29. The method of claim 28, wherein
a common first forbidden zone (90) resp. a common first allowed zone (91) is comprised in each respective forbidden zone (90) resp. allowed zone (91) attributed a target object (3) of the first group, and/or
a common second forbidden zone (90) resp. a common second allowed zone (91) is comprised in each respective forbidden zone (90) resp. allowed zone (91) attributed to a target object (3) of the second group.

30. The method of claim 25, wherein the forbidden zone (90) and/or an allowed zone (91) attributed to a target object (3) depends on the facing direction of a person (30,40), wherein the person (30,40) preferably
comprises at least one body part that is the target object (3), and/or
comprises at least one body part that is attributed to the target object (3) as a reference object (4).
